(19) United States
(12) Patent Application Publication
(10) Pub. No.: US 2024/0325650 A1
(43) Pub. Date: Oct. 3, 2024

(54) MEDICAL SYRINGE AND METHOD FOR REDUCING MEDICATION WASTE

(71) Applicant: Becton, Dickinson and Company, Franklin Lakes, NJ (US)

(72) Inventors: Praveen Nalawade, Belgaum (IN); Hemant Vilas Belsare, Pune (IN)

(73) Assignee: Becton, Dickinson and Company, Franklin Lakes, NJ (US)

(21) Appl. No.: 18/130,023

(22) Filed: Apr. 3, 2023

(51) Int. Cl. A61M 5/31; A61M 5/315

(52) U.S. Cl. CPC A61M 5/3134; A61M 5/31513

(57) ABSTRACT

A medical syringe reduces medication waste that dispensing medication remaining within the syringe's Luer tip. The syringe plunger incorporates a ramrod with a jag on its distal end that is inserted through the Luer tip's lumen when the plunger is fully advanced into the syringe barrel. A center through bore of a syringe stopper is slidably coupled to the ramrod in fluid-sealed abutting contact. The center bore is initially oriented on the ramrod over the jag. Medication is aspirated into the syringe while the stopper is in its initial, axial position. The last, dose of medication is dispensed by advancing with the plunger with sufficient pressure to abut the stopper into contact with and translate the ramrod jag through the lumen of the Luer tip, thereby dispensing medication that was retained within the lumen.

### TECHNICAL FIELD

The present disclosure generally relates to a medical syringe that maximizes use of medication fluid inside the syringe and reduces medication waste.

### BACKGROUND

After medication dose administration, known medical syringes retain residual medication fluids in their Luer syringe tip, needle hub, and needle lumen. A great majority of the residual medication is retained in so-called “dead space” within the syringe tip and the needle hub. It is desirable to reduce medication waste by emptying the “dead space” during dosage administration.

FIG. 1 depicts a known prior art, 1 ml dosage capacity, medical syringe 10, having a syringe barrel 12, and a syringe Luer tip 16 on a distal end 14 of the barrel. The Luer tip defines a tip lumen 18 that is in fluid communication with a medication chamber defined within the syringe barrel 12. A translatable syringe plunger 20 is coupled to a syringe stopper 22. A distal tip 24 of the syringe stopper is in abutting orientation with the distal end 14 and the lumen 18 of the Luer tip 16 of the syringe barrel 12. A needle hub 26 is coupled to the syringe Luer tip 16. The needle hub 26 retains a cup-shaped needle ferrule 28 in opposed orientation to and in fluid communication with the tip lumen 18. The needle ferrule 28 retains the hollow syringe needle 30, with the needle lumen 32 in fluid communication with the needle ferrule 28, the Luer tip lumen 18 and the syringe cavity.

The syringe 10 has dispensed all medication previously retained within the syringe barrel 12, because the distal tip 24 of the syringe stopper 22 is in abutting orientation with the distal end 14 and the lumen 18 of the Luer tip 16. However, residual medication remains within three dead space zones distal the syringe stopper's distal tip 24, shown in cross-hatch pattern in FIG. 1. Namely, the needle hub dead space 34, the needle lumen dead space 36, and Luer-tip lumen's dead space 38. In the 1 ml capacity syringe 10, the needle lumen dead space has a relatively low volume of approximately 0.005 ml, needle hub dead space 34 is a more significant volume of approximately 0.030 ml, and the Luer tip dead space 38 has the largest volume of approximately 0.033 ml. Combined dead space in the 1 ml dosage capacity syringe 12 is approximately 0.068 ml or about 6.8 percent of the medicine dose. In order to administer a full 1 ml dose of medication with the syringe 12, 1.068 ml of the medication must be aspirated into the syringe to compensate for the 6.8 percent of wasted medication.

### SUMMARY

Medical syringes of the present disclosure reduce medication waste by dispensing residual medication left in the Luer tip of the syringe barrel. A ramrod incorporated on the distal tip of the syringe plunger advances through the lumen within the Luer tip of the syringe barrel and dispenses residual medication out of the tip. In some embodiments the syringe also dispenses residual medication left in the needle hub of the syringe, such as in the needle ferrule. In clinical applications where multiple doses of medication, such as vaccines, are dispensed from a syringe, embodiments of medical syringes of the present disclosure dispense a more complete, last remaining dose that includes a significant volume of the fluid that was retained within the Luer tip and the needle hub.

One aspect of the present disclosure pertains to a medical syringe, which includes a syringe barrel, an interior sidewall, a proximal open end, a distal end, and a syringe cavity defined within the interior side wall and the distal end of the syringe barrel. The syringe barrel has a barrel Luer tip on its distal end. The barrel Luer tip defines a tip outlet and a lumen therethrough. The tip lumen is establishes fluid communication between the syringe cavity and the tip outlet and has a cross-sectional area. The syringe has a plunger, with a plunger shaft. A distal end of the plunger shaft is inserted within and is translatable within the syringe cavity. A ramrod has a proximal end rigidly coupled to the distal end of the plunger shaft, and a ramrod axial centerline that is coaxial with the axial centerline of the syringe. The ramrod is insertable within the Luer tip lumen to at least the tip outlet. A distal end of the ramrod defines a jag with a jag tip having a distal face. The jag has a cross-sectional area measured perpendicular to the axial centerline of the lumen of the Luer tip that is smaller than the corresponding cross-sectional area of the tip lumen. A syringe stopper is slidably coupled to the ramrod by a center bore circumscribing and in biased, fluid-sealed abutting contact with an outer circumferential surface of the ramrod. The syringe stopper has a distal axial end in opposed orientation with the distal end of the syringe barrel, which together with the barrel defines distal and proximal axial boundaries of the syringe cavity therebetween. A proximal end of the stopper is in opposed orientation with the distal end of the plunger shaft, and an outer circumferential surface of the syringe stopper is in fluid-sealed, abutting contact with the interior sidewall of the syringe barrel. The syringe stopper is oriented in an initial axial position on the ramrod, with its center bore in biased contact with the jag upon initial insertion of the plunger into the syringe barrel, as well as during aspiration or delivery of fluid into or out of the syringe cavity. Upon full advancement of the syringe plunger into the syringe barrel, the syringe stopper is translated into a final axial position along the ramrod, with the distal axial end of the syringe stopper in abutting contact with distal end of the syringe barrel and the ramrod jag captured within the lumen of the Luer tip, with the jag tip at least axially aligned with the outlet of the Luer tip. Thus, residual medication that was in the lumen is dispensed, providing a more complete medication dosage.

Another aspect of the present disclosure pertains to a medical syringe, having a syringe barrel with an axial centerline, an interior sidewall circumscribing the axial centerline, a proximal open end, a distal end, and a syringe cavity defined within the interior sidewall and the distal end of the syringe barrel. The syringe barrel has a barrel Luer tip on its distal end that defines a tip outlet and a lumen therethrough that is coaxial with the syringe axial centerline. The tip lumen is in fluid communication between the syringe cavity and the tip outlet and has a cross-sectional area measured perpendicular to the axial centerline of the syringe barrel. The syringe includes a plunger having a plunger shaft coaxial with the axial centerline of the syringe barrel, with a distal end of the plunger shaft inserted within and translatable within the syringe cavity. The plunger shaft has at least one spacer ring radially outwardly projecting from an outer circumferential surface of the plunger shaft proximate the distal end of the shaft, for maintaining coaxial alignment of the plunger and the interior sidewall of the syringe barrel. A ramrod has a proximal end rigidly coupled to the distal end of the plunger shaft, coaxial with the axial centerline of the syringe. The ramrod is insertable within the Luer tip lumen to at least the tip outlet. The ramrod has a jag with a jag tip on its distal end. The jag tip has a distal face that projects axially out of the lumen outlet when the syringe plunger is fully advanced into the syringe barrel. The jag has a primary lug and at least one secondary lug projecting radially outwardly relative to the syringe barrel centerline, the primary lug has a cross-sectional area measured perpendicular to the axial centerline of the syringe that is smaller than the corresponding cross-sectional area of the tip lumen, and at least one secondary lug having a cross-sectional area smaller than that of the primary lug. A syringe stopper is slidably coupled to the ramrod, with a center bore circumscribing and in biased, fluid-sealed abutting contact with an outer circumferential surface of the ramrod. The center bore of the syringe stopper defines a radially outwardly projecting recess for capturing the primary lug therein and additional recesses for capturing a corresponding secondary lug therein, the syringe stopper has a distal axial end in opposed orientation with the distal end of the syringe barrel, which with the syringe barrel defining distal and proximal axial boundaries of the syringe cavity therebetween. A proximal end of the stopper is in opposed orientation with the distal end of the plunger shaft, and an outer circumferential surface of the syringe stopper is in fluid-sealed abutting contact with the interior sidewall of the syringe barrel. The syringe stopper is oriented in an initial axial position on the ramrod, with the respective recesses of its center bore in biased contact with the corresponding lugs of the jag upon initial insertion of the plunger into the syringe barrel, as well as during aspiration or delivery of fluid into or out of the syringe cavity. Upon full advancement of the syringe plunger into the syringe barrel, the syringe stopper is translated into a final axial position along the ramrod, with the distal axial end of the syringe stopper in abutting contact with distal end of the syringe barrel. In this final position, the ramrod jag is captured within the lumen of the Luer tip, with the jag tip axially aligned with the outlet of the Luer tip and the distal face of the jag tip projecting axially out of the lumen outlet.

Yet another aspect of the present disclosure pertains to a method for dispensing medication remaining within a lumen of a Luer tip of a medical syringe. The syringe used to perform this method embodiment has a syringe barrel with a barrel Luer tip. The Luer tip has a tip outlet and a lumen therethrough. A plunger is oriented in the syringe barrel in communication with the tip outlet. The plunger has a plunger shaft and a ramrod having a proximal end rigidly coupled to the distal end of the plunger shaft A jag on a distal tip of the ramrod is insertable through the Luer tip lumen to at least the tip outlet. A syringe stopper is slidably coupled to the ramrod by a center bore circumscribing and in biased, fluid-sealed abutting contact with the ramrod. The syringe stopper is oriented in an initial axial position on the ramrod, with its center bore in biased contact over the jag. A needle hub is coupled to the Luer tip. The hub has a cup-shaped needle ferrule facing the outlet of and in fluid communication with the Luer tip, and a hollow needle coupled to and in fluid communication with the needle ferrule. When performing this method embodiment at least one dose of medication is aspirated into the syringe without translating the stopper from its initial position. A needle hub is coupled to the Luer tip, if not done prior to the aspiration of medication. At least one dose of medication is dispensed with the syringe, by advancing with the plunger with sufficient pressure to abut the stopper into contact with and translate the ramrod jag through the lumen of the Luer tip, so that the jag tip is axially aligned with the outlet, thereby dispensing medication that was retained within the lumen.

### BRIEF DESCRIPTION OF THE DRAWINGS

Exemplary embodiments of the disclosure are further described in the following detailed description in conjunction with the accompanying drawings, in which:

FIG. 1 is an axial, cross-sectional view of a prior art syringe, coupled to a needle hub;

FIG. 1A is an enlargement of the needle hub and Luer tip portions of the prior art syringe of FIG. 1;

FIG. 2 is an axial, cross-sectional view of an embodiment of a syringe of the present disclosure, coupled to a needle hub, showing the syringe plunger, ramrod of the syringe plunger, and plunger stopper in a partially advanced position within the syringe barrel;

FIG. 3 is an axial, cross-sectional view of the syringe of FIG. 2, with the plunger and ramrod further advanced so that the plunger stopper is in abutting contact with a distal end of the syringe barrel and the lumen of its Luer tip;

FIG. 3A is an enlargement of the plunger stopper and the ramrod of the syringe of FIG. 3;

FIG. 4 is an axial, cross-sectional view of the syringe of FIG. 3, with the plunger fully advanced, so that the ramrod of the plunger is fully advanced into the lumen of the Luer tip;

FIG. 4A is an enlargement of the needle hub, Luer tip and advanced ramrod portions of the syringe of FIG. 4;

FIG. 5 is an exploded view of the syringe of FIG. 2; and

FIG. 5A is an enlargement of the ramrod and jag of the syringe of FIG. 5.

To facilitate understanding, identical reference numerals have been used, where possible, to designate identical elements that are common to the figures. The figures are not drawn to scale.

### DETAILED DESCRIPTION

A medical syringe constructed in accordance with aspects of the present disclosure reduces wasted medication by displacing the medication otherwise retained within dead space of its Luer tip with a ramrod on a distal end of the syringe plunger. When the plunger is fully advanced within the syringe barrel, the ramrod advances through the lumen within the Luer tip of the syringe barrel and dispenses residual medication out of the tip. In some embodiments the syringe ramrod also displaces by medication in the needle hub, such as in the needle ferrule.

Aspects of the present disclosure also relate to a method for dispensing medication remaining within a lumen of a Luer tip of a medical syringe by advancing a ramrod incorporated on the distal tip of the syringe plunger through the lumen within the Luer tip of the syringe barrel In some embodiments, the medical syringe used to dispense the residual medication has a syringe barrel having a barrel Luer tip with a tip outlet and a lumen therethrough. A plunger oriented in the syringe barrel is in communication with the tip outlet. The plunger has a plunger shaft and a ramrod having a proximal end rigidly coupled to the distal end of the plunger shaft. A jag on a distal tip of the ramrod is insertable through the Luer tip lumen to at least the tip outlet. A syringe stopper is slidably coupled to the ramrod by a center bore circumscribing and in biased, fluid-sealed abutting contact with the ramrod. Initially, the syringe stopper is oriented in an initial axial position on the ramrod, with its center bore in biased contact over the ramrod's jag. A needle hub is coupled to the Luer tip. The hub has a cup-shaped needle ferrule facing the outlet of and in fluid communication with the Luer tip, and a hollow needle coupled to and in fluid communication with the needle ferrule.

In practicing this embodiment of the residual medication dispensing method, at least one dose of medication is aspirated into the syringe without translating the stopper from its initial position, in contact with the jag of the ramrod. The needle hub is coupled to the Luer tip, if not done prior to the aspiration of medication. At least one dose of medication is dispensed from the syringe by advancing with the plunger with sufficient pressure to abut the stopper into contact with and translate the ramrod jag through the lumen of the Luer tip, so that the jag tip is axially aligned with the outlet; this dispenses residual medication that was retained within the lumen.

In this disclosure, a convention is followed wherein the distal end of the device is the end closest to a patient and the proximal end of the device is the end away from the patient and closest to a practitioner.

With respect to terms used in this disclosure, the following definitions are provided. As used herein, the use of “a,” “an,” and “the” includes the singular and plural. As used herein, the term “catheter related bloodstream infection” or “CRBSI” refers to any infection resulting from the presence of a catheter or IV line.

As used herein, the term “Luer connector” refers to a connection collar that is the standard way of attaching syringes, catheters, hubbed needles, IV tubes, etc. to each other. The Luer connector consists of male and female interlocking tubes, slightly tapered to hold together better with even just a simple pressure/twist fit. Luer connectors can optionally include an additional outer rim of threading, allowing them to be more secure. The Luer connector male end is generally associated with a flush syringe and can interlock and connect to the female end located on the vascular access device (VAD) Luer connector also has a distal end channel that releasably attaches the Luer connector to the hub of a VAD, and a proximal end channel that releasably attaches the Luer connector to the barrel of a syringe.

As used herein, ISO 80369-7:2016 defines a specification for standard Luer connectors including a 6% taper between the distal end and the proximal end. A male standard luer connector increases from the open distal end to the proximal end. A female standard luer connector decreases from the open proximal end to the distal end. According to ISO 80369-7:2016, a male standard luer connector has an outer cross-sectional diameter measured 0.75 mm from the distal end of the tip of between 3.970 mm and 4.072 mm. The length of the male standard luer taper is between 7.500 mm to 10.500 mm. The outer cross-sectional diameter measured 7.500 mm from the distal end of the tip is between 4.376 mm and 4.476 mm. As used herein, the phrases “male standard luer connector” and “female standard luer connector” shall refer to connectors having the dimensions described in ISO 80369-7, which is hereby incorporated by reference in its entirety.

As would be readily appreciated by skilled artisans in the relevant art, while descriptive terms such as “tip”, “hub”, “thread”, “protrusion/insert”, “tab”, “slope”, “wall”, “top”, “side”, “bottom” and others are used throughout this specification to facilitate understanding, it is not intended to limit any components that can be used in combinations or individually to implement various aspects of the embodiments of the present disclosure.

As used herein an “axial centerline” is a linear axis passing through a longest dimension of a medical syringe component, such as an internal cavity of a syringe barrel, a syringe plunger or a lumen of a syringe's Luer tip. In some embodiments of the present disclosure, the axial centerline is laterally offset from a centroidal axis of a component. For example, a coaxially aligned Luer tip lumen, ramrod and plunger of a syringe barrel are laterally offset from the centroidal axis of a syringe barrel. While embodiments of syringes of the present disclosure are depicted with cylindrical, coaxial cross sections, in other embodiments the components have other cross sections, e.g., triangular, rectangular and/or asymmetrical cross sections.

Before describing several exemplary embodiments of the disclosure, it is to be understood that the disclosure is not limited to the details of construction or process steps set forth in the following description. The disclosure is capable of other embodiments and of being practiced or being conducted in many ways.

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

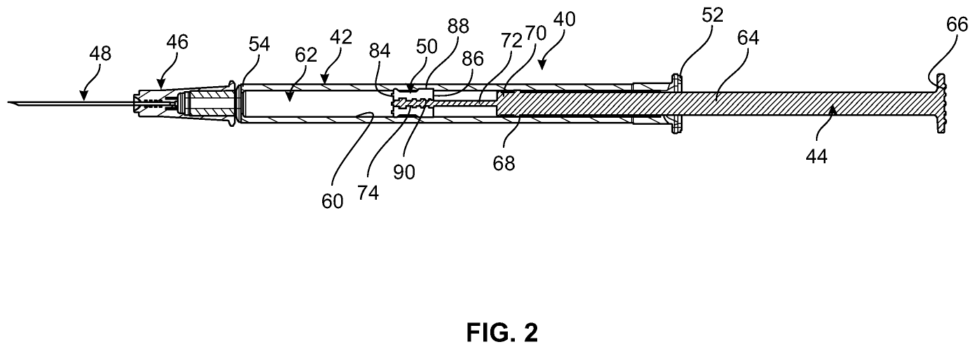

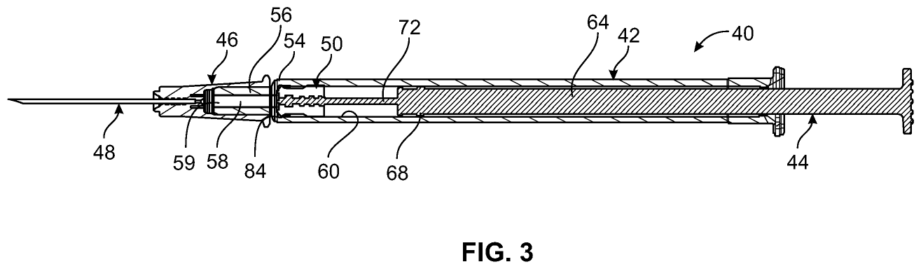

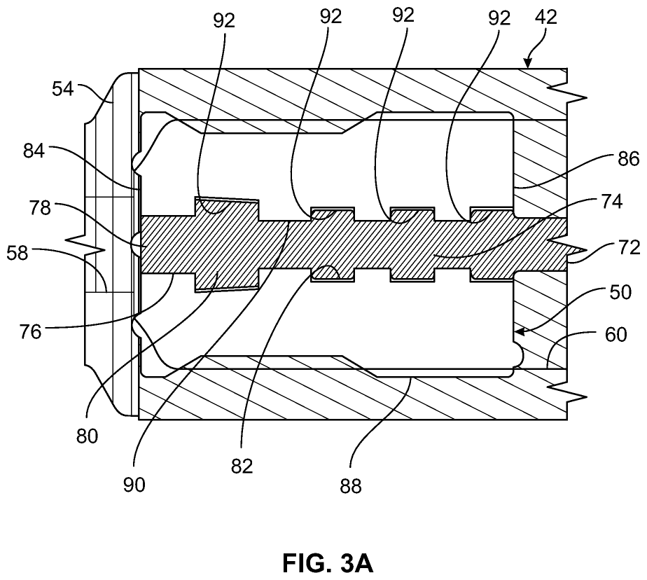

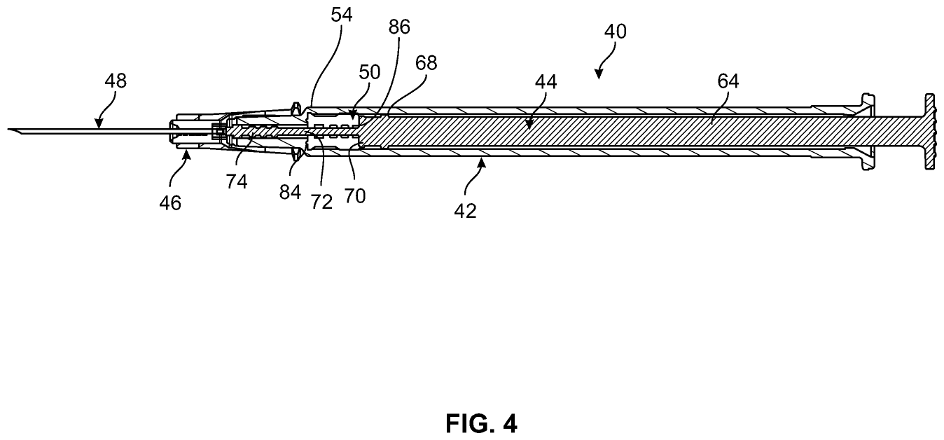

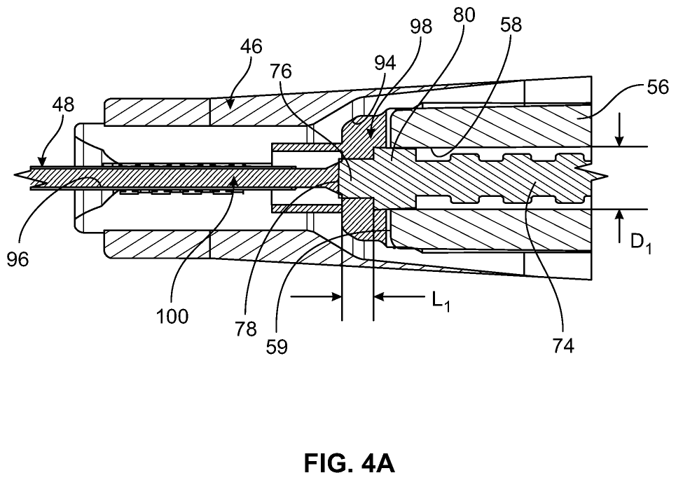

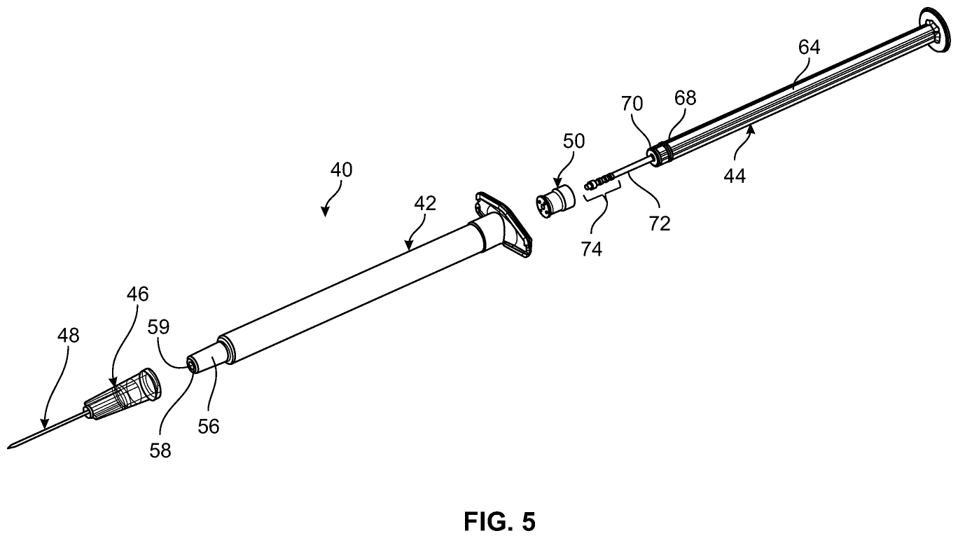

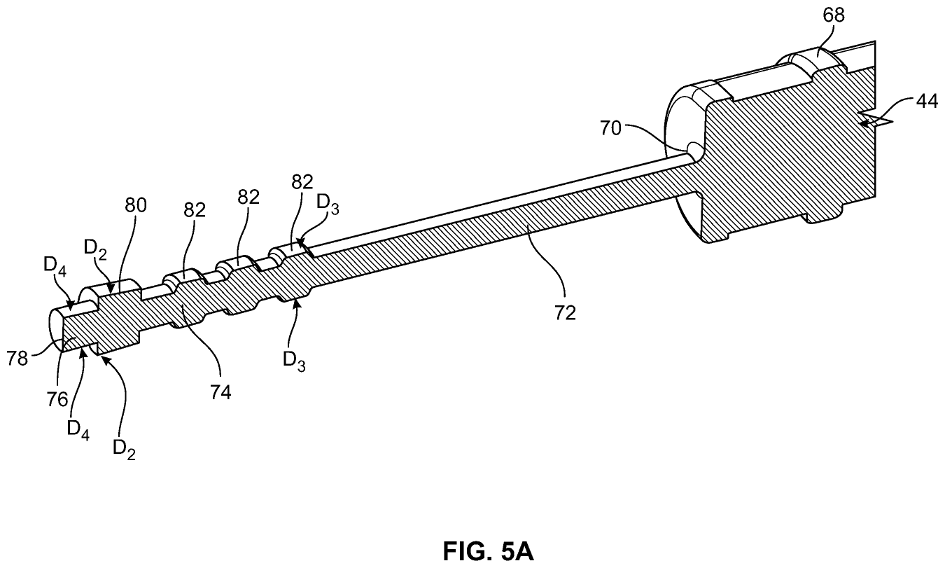

An exemplary implementation of an embodiment of present disclosure is depicted in FIGS. 2-5A. Referring to FIGS. 2 and 3, a syringe 40 includes a syringe barrel 42, a plunger 44 translatable within the syringe barrel, a needle hub 46, a hollow needle 48 coupled to the needle hub and a syringe stopper 50 coupled to the plunger 44. The syringe 40 and the syringe barrel 42 define an axial centerline that is coaxial with respective centerlines of the plunger 44, the needle hub 46, needle 48 and the stopper 50. The syringe barrel 42 has grasping tabs 52 on its open proximal end. A distal end 54 of the syringe barrel 42 incorporates a barrel Luer tip 56 with an internal Luer tip lumen 58 therethrough that is coaxial with the syringe 40 axial centerline. As shown in FIGS. 4A, the tip lumen 58 has a cylindrical profile, with a diameter D₁ whose cross-sectional area is measured perpendicular to the axial centerline of the syringe 40. The tip lumen 58 terminates in a distal tip outlet 59 as shown in FIGS. 3 and 5. The syringe barrel 42 has an interior sidewall 60 that defines a barrel cavity 62, along with its distal end 54 and the stopper 50. The tip lumen 58 establishes fluid communication between its tip outlet 59 and the syringe cavity 62.

Referring to FIGS. 2, 3A and 5A, the plunger 44 has a plunger shaft 64 whose centerline coaxial with the axial centerline of the syringe 40 and the syringe barrel 42. A proximal end of the plunger shaft 64 defines finger tabs 66. At least one spacer ring 68 projects radially outwardly from an outer circumferential surface of the plunger shaft 64, for maintaining coaxial alignment of the plunger shaft and the interior sidewall 60 of the syringe barrel 42. At least one spacer ring 68 is oriented proximate the distal end 70 of the plunger shaft 64.

A ramrod 72 has a proximal end rigidly coupled to the distal end 70 of the plunger shaft 64, coaxial with the latter's axial centerline, and is insertable within the Luer tip lumen 58 to at least the tip outlet 59. In some embodiments, the ramrod 72 is not insertable fully to the tip outlet 59. A distal end of the ramrod 72 defines a jag 74, with a jag tip 76 and a distal face 78 of which faces the lumen 58 of the Luer tip 56. As shown in FIGS. 4 and 4A, jag tip 76 has an axial length Li selected so that the distal face 78 of the jag tip 76 projects axially out of the lumen outlet 59 when the syringe plunger 44 is fully advanced into the syringe barrel.

Referring to FIGS. 4A and 5A, the jag 74 has a primary lug 80 and at least one secondary 82 lug projecting radially outwardly relative to the ramrod 72 axial centerline or that of the syringe barrel. In the exemplary non-limiting embodiment shown in FIGS. 4A and 5A, the jag 74 has a primary lug 80 and three secondary 82 lugs projecting radially outwardly relative to the ramrod 72 axial centerline or that of the syringe barrel. While the Luer tip lumen 58, the ramrod 72 and the lugs 80, 82 have mating, complementary cylindrical profiles and cross sections, in other embodiments they have other complimentary cross sections, such as square, triangular, oval or asymmetrical cross sections. The primary lug 80 has a diameter D₂ with a cross-sectional area measured perpendicular to the axial centerline of the ramrod 72 that is smaller than the diameter D₁ of the complementary, corresponding cross-sectional area of the tip lumen 58. Clearance gap between the complementary diameters D₁ and D₂ is selected so that the ramrod 72 does not bind within the tip lumen 58 but nonetheless clears fluid out of the lumen. In FIG. 5A, the secondary lugs 82 have a diameter D₃ and cross-sectional area smaller than that of the primary lug 80 to inhibit bending of the ramrod 72 within the tip lumen 58 without binding therein. The jag tip 76 has a diameter D₄.

Referring to FIGS. 2, 3 and 3A, the syringe stopper 50 has a distal axial end 84 that faces and is in opposed orientation with the barrel distal end 54, a proximal end 86 that faces the distal axial end 70 of the plunger shaft 64, and outer circumferential surface 88 that is in fluid-sealed, abutting contact with the interior sidewall 60 of the syringe barrel 42. The barrel's interior sidewall 60, along with its distal end 54 and the distal axial end 84 of the syringe stopper 50 define the barrel cavity 62, whose volume varies with axial position of the plunger stopper 50 and its coupled plunger 44 within the syringe barrel 42. The syringe stopper 50 is constructed of a pliable material (e.g., an elastomer) and is slidably coupled to the ramrod 72, by a an inner or center bore 90 that circumscribes and is biased, fluid-sealed abutting contact with an outer circumferential surface of the ramrod. The center bore 90 defines a radially outwardly projecting inner recesses 92, for capturing the primary lug 80 and the secondary lugs 80 therein.

As shown in FIG. 2, the syringe stopper 50 is oriented in an initial axial position on the jag 74 ramrod 72, with the respective recesses 92 of its center bore 90 in biased contact with the corresponding lugs 80, 82 of the jag 74, upon initial insertion of the plunger 44 into the syringe barrel 42, as well as during aspiration or delivery of fluid into or out of the syringe cavity 62. In some embodiments, multiple doses of a drug are delivered with the same syringe 40, by exchanging fresh needle hubs 46 and their attached needles 48. Referring now to FIGS. 3 and 3A, the syringe stopper 50 remains engaged with the jag 74 during advancement of the plunger 44 during drug delivery until the distal axial end 84 of the syringe stopper is in abutting contact with distal end 54 of the syringe barrel 42. Axial force applied to the plunger 44 necessary to retract or advance its coupled plunger stopper 50 within the syringe barrel 42 for aspiration or delivery of a fluid drug is less than the shearing axial force needed to separate mating contact of the stopper recesses 92 the jag lugs 80, 82.

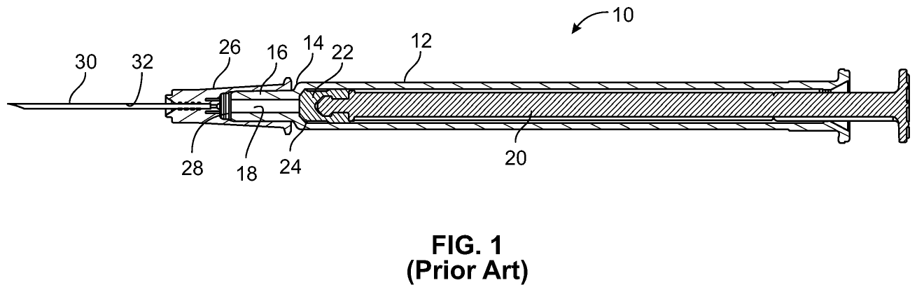

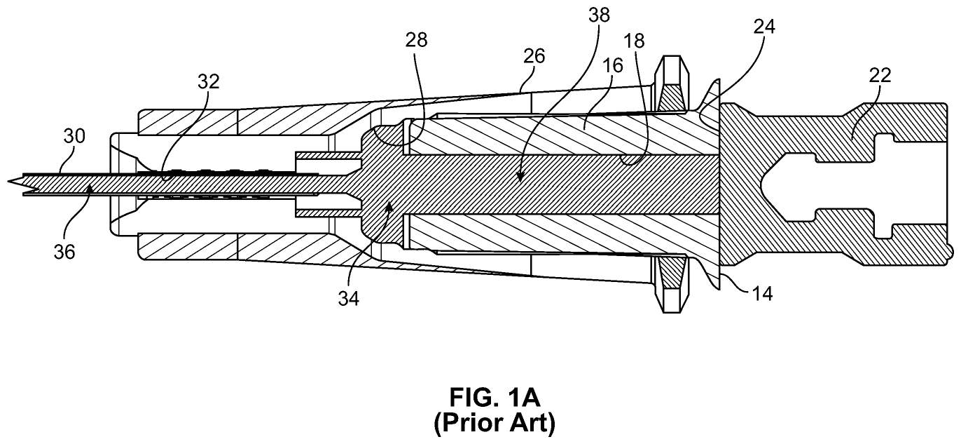

In FIG. 3, after full advancement of the syringe plunger 44 into the syringe barrel 42, where the distal axial end 84 of the syringe stopper 50 is in abutting contact with distal end 54 of the syringe barrel 42 residual medication remains in the lumen 58 of the syringe Luer tip 56, the needle hub 46 and the needle 48, as was the example of the prior art syringe 10 of FIG. 1. The syringe 40 of the present disclosure discharges residual medication, reducing drug waste and providing a complete medication dose to the patient, by enabling the medical provider to advance the ramrod 72 through the Luer tip lumen 58.

In FIGS. 4 and 4A, after the syringe stopper 50 is translated into a final axial position along the ramrod 72, with the distal axial end 84 of the syringe stopper 50 in abutting contact with distal end 54 of the syringe barrel 42, additional axial shearing force is exerted on the plunger 44. The additional axial force advances the ramrod 72 into the lumen 58 of the Luer tip 56, by separating the lugs 80, 82 out of the recesses 92 of the stopper. Upon shearing separation, this additional applied axial force advances the jag 74 through the outlet 59 of the Luer tip 56. The distal face 78 of the jag tip 76 projects into a cup-shaped needle ferrule 94 of the needle hub 46, so that the distal face 78 of the jag tip approaches the needle lumen 96 of the needle 48. The fully advanced ramrod 72 displaces residual fluid medication out of the lumen 58 of the syringe's Luer tip 56 and partially displaces medication in the remaining dead space 98 of the needle ferrule 94. A relatively small amount of medication remains in the needle ferrule's dead space 98 as well as an even smaller amount of medication within the needle lumen's dead space 100.

The following non-limiting example demonstrates residual medication reduction principles of the syringe according to one or more embodiments of the disclosure. A 1 ml dosage capacity embodiment of the syringe 50 significantly reduces medication dead space and medication waste compared to the known, comparable capacity syringe 10 (approximately 0.068 ml wasted medication). The ramrod 72 of the syringe 50 dispenses all medication from the Luer tip lumen 58 and reduces the needle hub dead space 98 to approximately 0.01 ml; the needle lumen dead space 100 remains at 0.005 ml. Total wasted medication remaining in the syringe 50 embodiment is reduced to approximately 0.015 ml, compared to the 0.068 ml wasted in the prior art, comparable capacity syringe 10 embodiment: an approximately 78 percent medication waste reduction.

The syringe embodiments disclosed herein are constructed from medical grade materials known to one skilled in the art. In some embodiments, described barrels, plungers and shafts are fabricated with polypropylene polymers. Seals are fabricated with fiber-filled polytetrafluoroethylene (PTFE) polymers. Stoppers are fabricated with polyisoprene polymers.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

What is claimed is:

1. A medical syringe, comprising:
a syringe barrel having an interior sidewall, a proximal open end, a distal end, a syringe cavity defined within the interior sidewall and the distal end of the syringe barrel, and a barrel Luer tip on the distal end of the syringe barrel, the barrel Luer tip defining a tip outlet and a lumen therethrough that defines a lumen axial centerline, the tip lumen establishing fluid communication between the syringe cavity and the tip outlet and having a cross-sectional area measured perpendicular to the lumen axial centerline;
a plunger having a plunger shaft, a distal end of the plunger shaft inserted within and translatable within the syringe cavity;
a ramrod having a proximal end rigidly coupled to the distal end of the plunger shaft, and a ramrod axial centerline that is coaxial with the lumen axial centerline, the ramrod insertable within the Luer tip lumen to at least the tip outlet;
a distal end of the ramrod defining a jag with a jag tip having a distal face, the jag having a cross-sectional area measured perpendicular to the lumen axial centerline that is smaller than the corresponding cross-sectional area of the tip lumen; and
a syringe stopper slidably coupled to the ramrod, having a center bore circumscribing and in biased, fluid-sealed abutting contact with an outer circumferential surface of the ramrod, the syringe stopper having a distal axial end in opposed orientation with the distal end of the syringe barrel, defining distal and proximal axial boundaries of the syringe cavity therebetween, a proximal end of the syringe stopper in opposed orientation with the distal end of the plunger shaft, and an outer circumferential surface of the syringe stopper in fluid-sealed abutting contact with the interior sidewall of the syringe barrel;
the syringe stopper oriented in an initial axial position on the ramrod, with its center bore in biased contact with the jag upon initial insertion of the plunger into the syringe barrel, as well as during aspiration or delivery of fluid into or out of the syringe cavity; and
upon full advancement of the syringe plunger into the syringe barrel, the syringe stopper translated into a final axial position along the ramrod, with the distal axial end of the syringe stopper in abutting contact with distal end of the syringe barrel and the ramrod jag captured within the lumen of the Luer tip, with the jag tip at least axially aligned with the outlet of the Luer tip.

2. The syringe of claim 1, further comprising jag tip defining at least one lug projecting radially outwardly relative to the ramrod axial centerline.

3. The syringe of claim 2, further comprising the center bore of the syringe stopper defining at least one radially outwardly projecting recess for capturing therein the at least one lug of the jag tip when the syringe stopper is in its initial axial position.

4. The syringe of claim 2, further comprising the jag tip defining a primary lug and at least one secondary lug having a cross-sectional area smaller than that of the primary lug.

5. The syringe of claim 4, further comprising the center bore of the syringe stopper defining a radially outwardly projecting recess for capturing the primary lug therein and additional recesses for capturing a corresponding secondary lug therein, when the syringe stopper is in its initial axial position.

6. The syringe of claim 2, further comprising the jag tip defining a distal face that projects axially out of the lumen outlet when the syringe plunger is fully advanced into the syringe barrel.

7. The syringe of claim 1, further comprising an outer circumferential surface of the plunger shaft defining at least one radially outwardly projecting spacer ring for maintaining coaxial alignment of the plunger and the interior sidewall of the syringe barrel.

8. The syringe of claim 7, further comprising the at least one spacer ring oriented proximate the distal end of the plunger shaft.

9. The syringe of claim 1, advancement force on the plunger shaft required to translate the syringe stopper from its initial to its final axial position is greater than force required to translate the syringe stopper during aspiration or dispensing of fluid into or out of the syringe cavity, prior to abutting contact of its distal end with the distal end of the syringe barrel.

10. The syringe of claim 1, further comprising a needle hub coupled to the barrel Luer tip, having a cup-shaped needle ferrule in opposed orientation with the outlet of the Luer tip, the jag tip of the ramrod oriented in the needle ferrule upon full advancement of the syringe plunger into the syringe barrel.

11. A medical syringe, comprising:
a syringe barrel having: an axial centerline, an interior sidewall circumscribing the axial centerline, a proximal open end, a distal end, and a syringe cavity defined within the interior sidewall and the distal end of the syringe barrel, a barrel Luer tip on the distal end of the syringe barrel, the barrel Luer tip defining a tip outlet and a lumen therethrough that is coaxial with the syringe axial centerline, the tip lumen in fluid communication between the syringe cavity and the tip outlet and having a cross-sectional area measured perpendicular to the axial centerline of the syringe barrel;
a plunger having a plunger shaft coaxial with the axial centerline of the syringe barrel, a distal end of the plunger shaft inserted within and translatable within the syringe cavity, at least one spacer ring radially outwardly projecting from an outer circumferential surface of the plunger shaft proximate the distal end of the shaft, for maintaining coaxial alignment of the plunger and the interior sidewall of the syringe barrel;
a ramrod having: a proximal end rigidly coupled to the distal end of the plunger shaft, coaxial with the axial centerline of the syringe and insertable within the Luer tip lumen to at least the tip outlet; a jag with a jag tip on a distal end of the ramrod, the jag tip having a distal face that projects axially out of the lumen outlet when the syringe plunger is fully advanced into the syringe barrel, the jag having a primary lug and at least one secondary lug projecting radially outwardly relative to the syringe barrel centerline, the primary lug having a cross-sectional area measured perpendicular to the axial centerline of the syringe that is smaller than the corresponding cross-sectional area of the tip lumen, and the at least one secondary lug having a cross-sectional area smaller than that of the primary lug; and
a syringe stopper slidably coupled to the ramrod, having a center bore circumscribing and in biased, fluid-sealed abutting contact with an outer circumferential surface of the ramrod, the center bore of the syringe stopper defining a radially outwardly projecting recess for capturing the primary lug therein and additional recesses for capturing a corresponding secondary lug therein, the syringe stopper having a distal axial end in opposed orientation with the distal end of the syringe barrel, defining distal and proximal axial boundaries of the syringe cavity therebetween, a proximal end in opposed orientation with the distal end of the plunger shaft, and an outer circumferential surface of the syringe stopper in fluid-sealed abutting contact with the interior sidewall of the syringe barrel;
the syringe stopper oriented in an initial axial position on the ramrod, with the respective recesses of its center bore in biased contact with the corresponding lugs of the jag upon initial insertion of the plunger into the syringe barrel, as well as during aspiration or delivery of fluid into or out of the syringe cavity; and
upon full advancement of the syringe plunger into the syringe barrel, the syringe stopper translated into a final axial position along the ramrod, with the distal axial end of the syringe stopper in abutting contact with distal end of the syringe barrel and the ramrod jag captured within the lumen of the Luer tip, with the jag tip axially aligned with the outlet of the Luer tip and the distal face of the jag tip projecting axially out of the lumen outlet.

12. The syringe of claim 11, advancement force on the plunger shaft required to translate the syringe stopper from its initial to its final axial position is greater than force required to translate the syringe stopper during aspiration or dispensing of fluid into or out of the syringe cavity, prior to abutting contact of its distal end with the distal end of the syringe barrel.

13. The syringe of claim 11, further comprising a needle hub coupled to the barrel Luer tip, having a cup-shaped needle ferrule in opposed orientation with the outlet of the Luer tip, the axial face of the jag tip of the ramrod oriented in the needle ferrule upon full advancement of the syringe plunger into the syringe barrel.

14. A method for dispensing medication remaining within a lumen of a Luer tip of a medical syringe, comprising:
providing a medical syringe with: a syringe barrel having a barrel Luer tip with a tip outlet and a lumen therethrough, a plunger oriented in the syringe barrel in communication with the tip outlet, the plunger having a plunger shaft and a ramrod having a proximal end rigidly coupled to a distal end of the plunger shaft, a jag on a distal tip of the ramrod insertable through the Luer tip lumen to at least the tip outlet, a syringe stopper slidably coupled to the ramrod by a center bore circumscribing and in biased, fluid-sealed abutting contact with the ramrod, the syringe stopper oriented in an initial axial position on the ramrod, with its center bore in biased contact over the jag, and a needle hub coupled to the Luer tip, the hub having a cup-shaped needle ferrule facing the outlet of and in fluid communication with the Luer tip, and a hollow needle coupled to and in fluid communication with the needle ferrule;
aspirating at least one dose of medication into the syringe without translating the stopper from its initial position;
coupling the needle hub to the Luer tip, if not done prior to the aspiration of medication; and
dispensing the at least one dose of medication by advancing with the plunger with sufficient pressure to abut the stopper into contact with and translate the ramrod jag through the lumen of the Luer tip, so that the jag tip is axially aligned with the outlet, thereby dispensing medication that was retained within the lumen.

15. The method for dispensing medication of claim 14, further comprising: dispensing the at least one dose of medication by advancing with the plunger with sufficient pressure to abut the stopper into contact with and translate the ramrod jag through the lumen of the Luer tip, so that the jag tip is in the needle ferrule, thereby dispensing medication that was retained within the needle ferrule and the lumen.
